(19) United States
(12) Patent Application Publication
(10) Pub. No.: US 2011/0071767 A1
(43) Pub. Date: Mar. 24, 2011

(54) Hepatotoxicity Molecular Models

(75) Inventors: Mark Porter, Gaithersburg, MD (US); Brandon Higgs, Gaithersburg, MD (US); Donna Mendrick, Gaithersburg, MD (US); Michael Elashoff, Gaithersburg, MD (US)

(73) Assignee: OCIMUM BIOSOLUNTIONS, INC., INDIANAPOLIS, INDIANA (IN)

(21) Appl. No.: 11/547,759

(22) PCT Filed: Apr. 7, 2005
(86) PCT No.: PCT/US05/11532

§ 371 Date: Jan. 13, 2008

(51) Int. Cl. G01N 33/52; G01N 33/48

(52) U.S. Cl. 702/20

(57) ABSTRACT

The present invention includes methods of predicting hepatotoxicity of test agents and methods of generating hepatotoxicity prediction models using algorithms for analyzing quantitative gene expression information. The invention also includes microarrays, computer systems comprising the toxicity prediction models, as well as methods of using the computer systems by remote users for determining the toxicity of test agents.

### RELATED APPLICATIONS

This application is entitled to priority pursuant to 35 U.S.C. §119(e) to U.S. Provisional Patent Application No. 60/559,949, which was filed on Apr. 7, 2004. This application is also related to U.S. Provisional Application No. 60/554,981, entitled “Methods for molecular toxicology modeling,” filed Mar. 22, 2004, PCT Application No. PCT/US03/03194, filed Jan. 31, 2003 and PCT Application No. PCT/US01/23872, filed Jul. 31, 2001, all of which are herein incorporated by reference in their entirety.

### SEQUENCE LISTING SUBMISSION ON COMPACT DISC

The Sequence Listing submitted concurrently herewith on compact disc under 37 C.F.R. §§1.821(c) and 1.821(e) is herein incorporated by reference in its entirety. Four copies of the Sequence Listing, one on each of four compact discs are provided. Copy 1, Copy 2 and Copy 3 are identical. Copies 1, 2 and 3 are also identical to the CRF. Each electronic copy of the Sequence Listing was created on Mar. 28, 2005 with a file size of 5995 KB. The file names are as follows: Copy 1—gene logic 5134-wo.txt; Copy 2—gene logic 5134-wo.txt; Copy 3—gene logic 5134-wo.txt.; and CRF—gene logic 5134-wo.txt.

### BACKGROUND OF THE INVENTION

The need for methods of assessing the toxic impact of a compound, pharmaceutical agent or environmental pollutant on a cell or living organism has led to the development of procedures which utilize living organisms as biological monitors. The simplest and most convenient of these systems utilize unicellular microorganisms such as yeast and bacteria, since they are the most easily maintained and manipulated. In addition, unicellular screening systems often use easily detectable changes in phenotype to monitor the effect of test compounds on the cell. Unicellular organisms, however, are inadequate models for estimating the potential effects of many compounds on complex multicellular animals, as they do not have the ability to carry out biotransformations.

The biotransformation of chemical compounds by multicellular organisms is a significant factor in determining the overall toxicity of agents to which they are exposed. Accordingly, multicellular screening systems may be preferred or required to detect the toxic effects of compounds. The use of multicellular organisms as toxicology screening tools has been significantly hampered, however, by the lack of convenient screening mechanisms or endpoints, such as those available in yeast or bacterial systems. Additionally, certain previous attempts to produce toxicology prediction systems have failed to provide the necessary modeling data and statistical information to accurately predict toxic responses (e.g., WO 00/12760, WO 00/47761, WO 00/63435, WO 01/32928, and WO 01/38579).

The pharmaceutical industry spends significant resources to ensure that therapeutic compounds of interest are not toxic to human beings. This process is lengthy as well as expensive and involves testing in a series of organisms starting with rats and progressing to dogs or non-human primates. Moreover, modeling methods for designing candidate pharmaceuticals and their synthesis in nucleic acid, peptide or organic compound libraries has increased the need for inexpensive, fast and accurate methods to predict toxic responses. Toxicity modeling methods based on nucleic acid hybridization platforms would allow the use biological samples from compound-exposed animal or cell culture samples, such as rats or rat hepatocyte cell cultures, to detect human organ toxicity much earlier than has been possible to date.

### SUMMARY OF THE INVENTION

The invention includes methods of predicting at least one toxic effect of a compound, comprising: detecting the level of expression in a tissue or cell sample exposed to the compound of two or more genes from Tables 1,2, 5 and 6; wherein differential expression of the genes in Tables 1,2, 5 and 6 is indicative of at least one toxic effect. The invention also includes methods of predicting at least one toxic effect of a test compound, comprising: preparing a gene expression profile from a liver cell or tissue sample exposed to the test compound; and comparing the gene expression profile to a database comprising quantitative gene expression information for at least one gene or gene fragment of Tables 1, 2 5 and 6 from a liver cell or tissue sample that has been exposed to at least one toxin and quantitative gene expression information for at least one gene or gene fragment of Tables 1, 2 5 and 6 from a control liver cell or tissue sample exposed to the toxin excipient, thereby predicting at least one toxic effect of the test compound.

In various aspects, the invention also includes methods of predicting at least one toxic effect of a test agent by comparing gene expression information from agent-exposed samples to a database of gene expression information from toxin-exposed and control samples (vehicle-exposed samples or samples exposed to a non-toxic compound or low levels of a toxic compound). These methods comprise providing or generating quantitative gene expression information from the samples, converting the gene expression information to matrices of fold-change values by a robust multi-array (RMA) algorithm, generating a gene regulation score for each gene that is differentially expressed upon exposure to the test agent by a partial least squares (PLS) algorithm, and calculating a sample prediction score for the test agent. This sample prediction score is then compared to a reference prediction score for one or more toxicity models.

In various aspects, the invention includes methods of creating a toxicity model. These methods comprise providing or generating quantitative nucleic acid hybridization data for a plurality of genes from at least one cell or tissue sample exposed to a toxin and at least one cell or tissue sample exposed to the toxin vehicle, converting the hybridization data from at least one gene to a gene expression measure, such as fold-change value, by a robust multi-array (RMA) algorithm, generating a gene regulation score from gene expression measure for the at least one gene by a partial least squares (PLS) algorithm, and generating a toxicity reference prediction score for the toxin, thereby creating a toxicity model.

The invention further provides a set of genes or gene fragments, listed in Table 2, from which probes can be made and attached to solid supports. These core genes serve as a preferred set of markers of liver toxicity and can be used with the methods of the invention to predict or monitor a toxic effect of a compound or to modulate the onset or progression of a toxic response.

In other aspects, the invention includes a computer system comprising a computer readable medium containing a toxicity model for predicting the toxicity of a test agent and software that allows a user to predict at least one toxic effect of a test agent by comparing a sample prediction score for the test agent to a toxicity reference prediction score for the toxicity model.

In further aspects of the invention, the gene expression information from test agent-exposed tissues or cells may be prepared transmitted via the Internet for analysis and comparisons to the toxicity models stored on a remote, central server. After processing, the user that sent the text files receives a report indicating the toxicity or non-toxicity of the test agent.

Tables

Table 1: Table 1 provides the GLGC identifier (fragment names from Table 2) in relation to the SEQ ID NO. and GenBank Accession number for each of the genes, gene fragments, or proteins listed in Table 2 (all of which are herein incorporated by reference and replicated in the attached sequence listing). The gene names and Unigene cluster titles are also included.

Table 2: Table 2 presents the PLS weight scores (index scores) for each gene from a series of liver toxicity models.

Table 3: Table 3 lists the toxins and negative control compounds used to build and train each toxicity model. The designation “1” for a particular compound in a particular model indicates that the compound was used on the toxicity/pathology (tox) side for training the model. The designation “−1” for a particular compound in a particular model indicates that the compound was used on the non-toxicity/no pathology (non-tox) side for training the model. The designation “0” for a particular compound in a particular model indicates that the compound was not used to train that model.

Table 4: Table 4 provides the protocols used for administration of each toxin to rats and indicates the time points at which rats were sacrificed and tissue samples collected.

Table 5: Table 5 lists the corresponding human genes or gene fragments, as well as genes and gene fragments of other species, for the genes and gene fragments of Tables 1 and 2. The Unigene cluster titles for these corresponding genes are also included.

Table 6: Table 6 supplies information concerning the metabolic pathways in which the genes and gene fragments of Tables 1 and 2 function.

### DETAILED DESCRIPTION

Many biological functions are accomplished by altering the expression of various genes through transcriptional (e.g. through control of initiation, provision of RNA precursors, RNA processing, etc.) and/or translational control. For example, fundamental biological processes such as cell cycle, cell differentiation and cell death are often characterized by the variations in the expression levels of groups of genes.

Changes in gene expression are also associated with the effects of various chemicals, drugs, toxins, pharmaceutical agents and pollutants on an organism or cells. For example, the lack of sufficient expression of functional tumor suppressor genes and/or the over expression of oncogene/protooncogenes after exposure to an agent could lead to tumorgenesis or hyperplastic growth of cells (Marshall (1991) Cell 64: 313-326; Weinberg (1991) Science 254:1138-1146). Thus, changes in the expression levels of particular genes (e.g. oncogenes or tumor suppressors) may serve as signposts for the presence and progression of toxicity or other cellular responses to exposure to a particular compound.

Monitoring changes in gene expression may also provide certain advantages during drug screening and development. Often drugs are screened for the ability to interact with a major target without regard to other effects the drugs have on cells. These cellular effects may cause toxicity in the whole animal, which prevents the development and clinical use of the potential drug.

The present inventors have examined tissue from animals exposed to the known hepatotoxins which induce detrimental liver effects, to identify changes in gene expression induced by these compounds. These changes in gene expression, which can be detected by the production of gene expression profiles, provide useful toxicity markers that can be used to monitor toxicity and/or toxicity progression by a test compound. Some of these markers may also be used to monitor or detect various disease or physiological states, disease progression, drug efficacy and drug metabolism.

Definitions

As used herein, “nucleic acid hybridization data” refers to any data derived from the hybridization of a sample of nucleic acids to a one or more of a series of reference nucleic acids. Such reference nucleic acids may be in the form of probes on a microarray or may be in the form of primers that are used in polymerization reactions, such as PCR amplification, to detect hybridization of the primers to the sample nucleic acids. Nucleic hybridization data may be in the form of numerical representations of the hybridization and may be derived from quantitative, semi-quantitative or non-quantitative analysis techniques or technology platforms. Nucleic acid hybridization data includes, but is not limited to gene expression data. The data may be in any form, including florescence data or measurements of florescence probe intensities from a microarray or other hybridization technology platform. The nucleic acid hybridization data may be raw data or may be normalized to correct for, or take into account, background or raw noise values, including background generated by microarray high/low intensity spots, scratches, high regional or overall background and raw noise generated by scanner electrical noise and sample quality fluctuation.

As used herein, “cell or tissue samples” refers to one or more samples comprising cell or tissue from an animal or other organism, including laboratory animals such as rats or mice. The cell or tissue sample may comprise a mixed population of cells or tissues or may be substantially a single cell or tissue type, such as liver hepatocytes or tissue. Cell or tissue samples as used herein may also be in vitro grown cells or tissue, such as primary cell cultures, immortalized cell cultures, cultured hepatocytes, cultured liver tissue, etc. Cells or tissue may be derived from any organ, including but not limited to, liver, kidney, cardiac, muscle (skeletal or cardiac) or brain.

As used herein, “test agent” refers to an agent, compound or composition that is being tested or analyzed in a method of the invention. For instance, a test agent may be a pharmaceutical candidate for which toxicology data is desired.

As used herein, “test agent vehicle” refers to the diluent or carrier in which the test agent is dissolved, suspended in or administered in, to an animal, organism or cells.

As used herein, “toxin vehicle” refers to the diluent or carrier in which a toxin is dissolved, suspended in or administered in, to an animal, organism or cells.

As used herein, a “gene expression measure” refers to any numerical representation of the expression level of a gene or gene fragment in a cell or tissue sample. A “gene expression measure” includes, but is not limited to, a fold change value.

As used herein, “at least one gene” refers to a nucleic acid molecule detected by the methods of the invention in a sample. The term “gene” as used herein, includes fully characterized open reading frames and the encoded mRNA as well as fragments of expressed RNA that are detectable by any hybridization method in the cell or tissue samples assayed as described herein. For instance, a “gene” includes any species of nucleic acid that is detectable by hybridization to a probe in a microarray, such as the “genes” of Table 1. As used herein, at least one gene includes a “plurality of genes.”

As used herein, “fold change value” refers to a numerical representation of the expression level of a gene, genes or gene fragments between experimental paradigms, such as a test or treated cell or tissue sample, compared to any standard or control. For instance, a fold change value may be presented as microarray-derived florescence or probe intensities for a gene or genes from a test cell or tissue sample compared to a control, such as an unexposed cell or tissue sample or a vehicle-exposed cell or tissue sample. An RMA fold change value as described herein is a non-limiting example of a fold change value calculated by methods of the invention.

As used herein, “gene regulation score” refers to a quantitative measure of gene expression for a gene or gene fragment as derived from a weighted index score or PLS score for each gene and the fold change value from treated vs. control samples.

As used herein, “sample prediction score” refers to a numerical score produced via methods of the invention as herein described. For instance, a “sample prediction score” may be calculated using the weighted index score or PLS score for at least one gene in a gene expression profile generated from the sample and the RMA fold change value for that same gene. A “sample prediction score” is derived from summing the individual gene regulation scores calculated for a given sample.

As used herein, “toxicity reference prediction score” refers to a numerical score generated from a toxicity model that can be used as a cut-off score to predict at least one toxic effect of a test agent. For instance, a sample prediction score can be compared to a toxicity reference prediction score to determine if the sample score is above or below the toxicity reference prediction score. Sample prediction scores falling below the value of a toxicity reference prediction score are scored as not exhibiting at least one toxic effect and sample prediction scores above the value if a toxicity reference prediction score are scored as exhibiting at least one toxic effect.

As used herein, a log scale linear additive model includes any log scale linear - model such as log scale robust multi-array analysis or RMA (Irizarry et al., Nucleic Acids Research 31(4) e15 (2003).

As used herein, “remote connection” refers to a connection to a server by a means other than a direct hard-wired connection. This term includes, but is not limited to, connection to a server through a dial-up line, broadband connection, Wi-Fi connection, or through the Internet.

As used herein, a “CEL file” refers to a file that contains the average probe intensities associated with a coordinate position, cell or feature on a microarray. See the Affymetrix GeneChip® Expression Analysis Technical Manual, which is herein incorporated by reference.

As used herein, a “gene expression profile” comprises any quantitative representation of the expression of at least one mRNA species in a cell sample or population and includes profiles made by various methods such as differential display, PCR, microarray and other hybridization analysis, etc.

As used herein, a “general toxicity model” refers to a model that is not limited to a specific pathology or mechanism. This category classifies compounds by their ability to induce toxicity in one or more species, including humans. Compounds that only cause toxicity in the rat are excluded by this definition.

As used herein, a “necrosis model” refers to a model based on pathology observed in multiple species defined by general cell death via non-energy dependent swelling. See Spraycar, M., ed. Stedman's Medical Dictionary. 26 ed. 1995, Williams & Wilkins: Baltimore.

As used herein, a “steatosis model,” also referred to as an “adiposis model,” is defined by the accumulation of fat in cell vacuoles as observed in multiple species.

As used herein, a “macrovesicular steatosis model” is a form of general steatosis (see above), defined by fatty cells that each contain one (or very few) large vesicle that displaces the nucleus as observed in multiple species.

As used herein, a “microvesicular steatosis model” is a form of general steatosis (see above), defined by fatty cells that each contain numerous small vesicles that do not displace the nucleus as observed in multiple species.

As used herein, a “cholestasis model” refers to a pathology that results from an impairment in bile flow as observed in multiple species. See Cotran, R. S., V. Kumar, and T. Collins, eds. Pathologic Basis of Disease. 6 ed. 1999, W. B. Saunders Company: Philadelphia.

As used herein, a “hepatitis model” refers to injury to the liver associated with an influx of acute or chronic inflammatory cells. The pathology is difficult to identify in rats, but is well documented and easily identified in mice and humans. See Fromenty, B., A. Berson, and D. Pessayre, Microvesicular steatosis and steatohepatitis: role of mitochondrial dysfunction and lipid peroxidation. J Hepatol, 1997. 26 Suppl 1: p. 13-22.

As used herein, a “carcinogenicity model” refers to the capability for induction of tumors in humans or animal models.

As used herein, a “genotoxic carcinogenicity model” refers to a mechanism by which compounds damage DNA and initiate a cascade of events that lead to tumor formation as observed in multiple species.

As used herein, a “non-genotoxic carcinogenicity model” refers to a mechanism by which compounds cause tumor formation in the absence of DNA damage as observed in multiple species.

As used herein, a “rat-specific non-genotoxic carcinogenicity model” refers to a mechanism by which compounds cause tumor formation in the absence of DNA damage. In this case, there is a subset of compounds that only cause this type of tumor formation in a rodent.

As used herein, a “peroxisome proliferation model” refers to a pathology or mechanism evidenced by an increase in the number of peroxisomes within cells. Humans are refractory to the development of this phenotype.

As used herein, an “inducer/liver enlargement model” refers to a pathology evidenced by an increase in liver size in general. The liver can increase in size due to either hypertrophy, an increase in size of the cells, or hyperplasia, an increase in cell number as observed in multiple species.

As used herein, a “human-specific model” refers not to a specific pathology or mechanism, but rather classifies compounds by their absence of toxicity in animal models, but presence of adverse events in human patients.

Methods of Generating Toxicity Models

To evaluate and identify gene expression changes that are predictive of toxicity, studies using selected compounds with well characterized toxicity may be used to build a model or database of the present invention. In the present studies, the following hepatotoxins were used to build one or more of the models of the invention: 17-α-ethynylestradiol (EE), 2-acetylaminofluorene (2-AAF), 3-methylcholanthrene, Abacavir, acetaminophen (APAP; paracetamol), acetylsalicylic acid (aspirin), allyl alcohol, Amineptine, Amiodarone, Amitriptyline, ANIT (1-naphthyl isothiocyanate), Eisai (Aricept™), Aroclor 1254, AY-25329, BI compound, Bicalutamide, bromobenzene, Bupropion, Carbamazepine, carbon tetrachloride (CCl₄), Ceftazidime, chloroform, Chlorpheniramine maleate (ChlorTrimeton), CI 1000, Ciprofibrate, Clofibrate, Colchicine, Compound A (AZ), Compound B (AZ), Sulindac, Cyproterone acetate, CZB777, Dantrolene, Demeclocycline, Diclofenac, diethylnitrosamine (DEN), Diflunisal, Diphenhydramine, Diquat, DMN (dimethylnitrosamine), dopamine, Epirubicin, Erythromycin estolate, ethanol, Etoposide, Famotidine (Pepcid AC), Felbamate, Fenofibrate, Flutamide, Gemfibrozil, Gentamicin, Hydrazine (Isoniazid), hydroxyurea, Indomethacin, Labetalol, L-ethionine, LPS (lipopolysaccharide), d-mannitol, Menadione, Metformin, Methapyrilene, Methotrexate, methyldopa, Lovastatin (Mevacor), Monocrotaline, Org 10000, Org 20000, Paraquat, Perhexilene, Phenacetin, phenobarbital, physostigmine, Plicamycin, Rifabutin, Rifampin, Rosiglitazone, Simvastatin, Stavudine, Streptomycin, Tacrine, Tamoxifen, TCDD (2,3,7,8-tetrachlorodibenzo-p-dioxin), Temozolomide, Tetracycline, thioacetamide, Valproate, Wy-14,643, Zidovudine and Zileuton. Methods used to prepare the models of the present invention include an RMA/PLS method (analysis of raw gene expression data by the robust multi-array average algorithm, with evaluation of predictive ability by the partial least squares algorithm).

In general, the models of the invention are built using cell and tissue samples that are analyzed after exposure to compounds known to exhibit at least one toxic effect. Low doses of these compounds, or the vehicles in which they were prepared, are used as negative controls. Compounds that are known not to exhibit at least one toxic effect may also be used as negative controls. The changes in gene expression levels in samples treated with the compound were considered to represent a specific toxic response, and the genes whose expression was up- or down-regulated upon treatment with the compound were classified as marker genes that may be used as indicators of a specific type of toxic response, i.e., a specific type of liver pathology. These marker genes may also be used to prepare reference gene expression profiles that characterize a specific hepatotoxic response. To train a toxicity model that is initially built from a database of gene expression information classified as showing a toxic response or not showing a toxic response, information from samples treated with some compounds is removed from the model, while information from samples treated with other compounds is retained. If the model with the retained information also retains the ability of the original model to distinguish between a toxic response and the lack of a toxic response in test samples compared to the model, the genes in the training model whose expression is up- or down-regulated are used to build a specific toxicity model. These genes are used on the tox side of the training model.

The toxins and negative control compounds used to build and train each toxicity model are shown in Table 3. The designation “1” for a particular compound in a particular model indicates that the compound was used on the toxicity/pathology (tox) side for training the model. Where a particular compound in a particular model has the designation of “−1”, the gene expression information from samples treated with that compound is considered to represent the absence of a toxic response or pathology. This information was used on the non-tox side, or negative control side, for training a model to produce a specific toxicity model. The genes analyzed in these samples are considered not to be markers of toxicity. Where a particular compound in a particular model has the designation “0,” the compound was not used to train that model.

In the present invention, a toxicity study or “tox study” comprises a set of cell or tissue samples that have been exposed to one or more toxins and may include matched samples exposed to the toxin vehicle or a low, non-toxic, dose of the toxin. As described below, the cell or tissue samples may be exposed to the toxin and control treatments in vivo or in vitro. In some studies, toxin and control exposure to the cell or tissue samples may take place by administering an appropriate dose to an animal model, such as a laboratory rat. In some studies, toxin and control exposure to the cell or tissue samples may take place by administering an appropriate dose to a sample of in vitro grown cells or tissue, such as primary rat or human hepatocytes. These samples are typically organized into cohorts by test compound, time (for instance, time from initial test compound dosage to time at which rats are sacrificed), and dose (amount of test compound administered). All cohorts in a tox study typically share the same vehicle control. For example, a cohort may be a set of samples from rats that were treated with acyclovir for 6 hours at a high dosage (100 mg/kg). A time-matched vehicle cohort is a set of samples that serve as controls for treated animals within a tox study, e.g., for 6-hour acyclovir-treated high dose samples the time-matched vehicle cohort would be the 6-hour vehicle-treated samples with that study.

A toxicity database or “tox database” is a set of tox studies that alone or in combination comprise a reference database. For instance, a reference database may include data from rat tissue and cell samples from rats that were treated with different test compounds at different dosages and exposed to the test compounds for varying lengths of time. A hepatotoxicity database is a set of hepatotoxicity studies that alone or in combination comprise a reference database.

RMA, or robust multi-array average, is an algorithm that converts raw fluorescence intensities, such as those derived from hybridization of sample nucleic acids to an Affymetrix GeneChip® microarray, into expression values, one value for each gene fragment on a chip (Irizarry et al. (2003), Nucleic Acids Res. 31(4):e15, 8 pp.; and Irizarry et al. (2003) “Exploration, normalization, and summaries of high density oligonucleotide array probe level data,” Biostatistics 4(2): 249-264). RMA produces values on a log2 scale, typically between 4 and 12, for genes that are expressed significantly above or below control levels. These RMA values can be positive or negative and are centered around zero for a fold-change of about 1. A matrix of gene expression values generated by RMA can be subjected to PLS to produce a model for prediction of toxic responses, e.g., a model for predicting liver or kidney toxicity. In a preferred embodiment, the model is validated by techniques known to those skilled in the art. Preferably, a cross-validation technique is used. In such a technique, the data is randomly broken into training and test sets several times until an acceptable model success rate is determined. Most preferably, such technique uses ⅔/⅓ cross-validation, where ⅓ of the data is dropped and the other ⅔ is used to rebuild the model.

PLS, or Partial Least Squares, is a modeling algorithm that takes as inputs a matrix of predictors and a vector of supervised scores to generate a set of prediction weights for each of the input predictors (Nguyen et al. (2002), Bioinformatics 18:39-50). These prediction weights are then used to calculate a gene regulation score to indicate the ability of each analyzed gene to predict a toxic response. As described in the examples, the gene regulation scores may then be used to calculate a toxicity reference prediction score.

From the nucleic acid hybridization data, a gene expression measure is calculated for one or more genes whose level of expression is detected in the nucleic acid hybridization value. As described above, the gene expression measure may comprise an RMA fold change value. The toxicity reference score=Σwᵢ R^{FC}^{ᵢ}. “i” is the index number for each gene in a gene expression profile to be evaluated. “wᵢ” is the PLS weight (or PLS score, see Table 2) for each gene. “R^{FC}^{ᵢ}” is the RMA fold-change value for the i^{th} gene, as determined from a normalized RMA matrix of gene expression data from the sample (described above). The PLS weight multiplied by the RMA fold-change value gives a gene regulation score for each gene, and the regulation scores for all the individual genes are added to give a toxicity reference prediction score for a sample or cohort of sample. A toxicity reference prediction score can be calculated from at least one gene regulation score, or at least about 5, 10, 25, 50, 100, 500 or about 1,000 or more gene regulation scores.

In one embodiment of the invention, a toxicology or toxicity model of the invention is prepared or created by the steps of (a) providing nucleic acid hybridization data for a plurality of genes from at least one liver cell or tissue sample exposed to a toxin and at least one liver cell or tissue sample exposed to the toxin vehicle; (b) converting the hybridization data from at least one gene to a gene expression measure; (c) generating a gene regulation score from gene expression measure for said at least one gene; and (d) generating a toxicity reference prediction score for the toxin, thereby creating a toxicity model. The gene expression measure may be a gene fold change value calculated by a log scale linear additive model such as RMA and the toxicity reference prediction score may be generated with PLS. The toxicity reference prediction score may then be added to a toxicity model or database and be used to predict at least one toxic effect of an unknown test agent or compound.

In another preferred embodiment, the model is validated by techniques known to those skilled in the art. Preferably, a cross-validation technique is used. In such a technique, the data is randomly broken into training and test sets several times until an acceptable model success rate is determined. Most preferably, such technique uses ⅔/⅓ cross-validation, where ⅓ of the data is dropped and the other ⅔ is used to rebuild the model.

Methods of Predicting Toxic Effects

The gene regulation scores and toxicity prediction scores derived from cell or tissue samples exposed to toxins may be used to predict at least one toxic effect, including the hepatotoxicity, renal toxicity or other tissue toxicity of a test or unknown agent or compound. The gene regulation scores and toxicity prediction scores from liver cell or tissue samples exposed to toxins may also be used to predict the ability of a test agent or compound to induce a tissue pathology, such as liver necrosis, in a sample. The toxicology prediction methods of the invention are limited only by the availability of the appropriate toxicity model and toxicology prediction scores. For instance, the prediction methods of a given system, such as a computer system or database of the invention, can be expanded simply by running new toxicology studies and models of the invention using additional toxins or specific tissue pathology inducing agents and the appropriate cell or tissue samples.

As used, herein, at least one toxic effect includes, but is not limited to, a detrimental change in the physiological status of a cell or organism. The response may be, but is not required to be, associated with a particular pathology, such as tissue necrosis. Accordingly, the toxic effect includes effects at the molecular and cellular level. Hepatotoxicity, for instance, is an effect as used herein and includes but is not limited to the pathologies of: cholestasis, genotoxicity/carcinogenesis, hepatitis, human-specific toxicity, induction of liver enlargement, steatosis, macrovesicular steatosis, microvesicular steatosis, necrosis, non-genotoxic/non-carcinogenic toxicity, peroxisome proliferation, rat non-genotoxic toxicity, and general hepatotoxicity.

In general, assays to predict the toxicity of a test agent (or compound or multi-component composition) comprise the steps of exposing a liver cell or tissue sample or population of cell or tissue samples to the test agent or compound, providing nucleic acid hybridization data for at least one gene from the test agent exposed cell or tissue sample(s), by, for instant, assaying or measuring the level of relative or absolute gene expression of one or more of the genes, such as one or more of the genes in Table 2, calculating a sample prediction score and comparing the sample prediction score to one or more toxicology reference scores (see Example 1).

Sample prediction scores may be calculated as follows: sample prediction score=Σwᵢ R^{FC}^{ᵢ}. “i” is the index number for each gene in a gene expression profile to be evaluated. “wᵢ” is the PLS weight (or PLS score) for each gene derived from a toxicity model. “R^{FC}^{ᵢ}” is the RMA fold-change value for the i^{th} gene, as determined from a normalized RMA matrix of gene expression data from the sample (described above). The PLS weight from a given model multiplied by the RMA fold-change value gives a gene regulation score for each gene, and the regulation scores for all the individual genes are added to give a prediction score for the sample.

Nucleic acid hybridization data may include any measurement of the hybridization, including gene expression levels, of sample nucleic acids to probes corresponding to about 2, 3, 4, 5, 6, 7, 8, 9, 10, 15, 20, 25, 30, 50, 75, 100, 200, 500, 1000 or more genes, or ranges of these numbers, such as about 2-10, about 10-20, about 20-50, about 50-100, about 100-200, about 200-500 or about 500-1000 genes. Nucleic acid hybridization data for toxicity prediction may also include the measurement of nearly all the genes in a toxicity model. “Nearly all” the genes may be considered to mean at least about 80% of the genes in any one toxicity model.

The methods of the invention to predict at least one toxic effect of a test agent or compound may be practiced by one individual or at one location, or may be practiced by more than one individual or at more than one location. For instance, methods of the invention include steps wherein the exposure of a test agent or compound to a cell or tissue sample(s) is accomplished in one location, nucleic acid processing and the generation of nucleic acid hybridization data takes place at another location and gene regulation and sample prediction scores calculated or generated at another location.

In another embodiment of the invention, cell or tissue samples are exposed to a test agent or compound by administering the agent to laboratory rats and nucleic acids are processed from selected tissues and hybridized to a microarray to produce nucleic acid hybridization data. The nucleic acid hybridization data is then sent to a remote server comprising a toxicology reference database and software that enables generation of individual gene regulation scores and one or more sample prediction scores from the nucleic acid hybridization data. The software may also enable to user to pre-select specific toxicity models and to compare the generated sample prediction scores to one or more toxicology reference scores contained within a database of such scores. The user may then generate or order an appropriate output product(s) that presents or represents the results of the data analysis, generation of gene regulation scores, sample prediction scores and/or comparisons to one or more toxicology reference scores.

Data, including nucleic acid hybridization data, may be transmitted to a server via any means available, including a secure direct dial-up or a secure or unsecured internet connection. Toxicology prediction reports or any result of the methods herein may also be transmitted via these same mechanisms. For instance, a first user may transmit nucleic acid hybridization data to a remote server via a secure password protected internet link and then request transmission of a toxicology report from the server via that same internet link.

Data transmitted by a remote user of a toxicity database or model may be raw, un-normalized data or may be normalized from various background parameters before transmission. For instance, data from a microarray may be normalized for various chip and background parameters such as those described above, before transmission. The data may be in any form, as long as the data can be recognized and properly formatted by available software or the software provided as part of a database or computer system. For instance, microarray data may be provided and transmitted in a cel file or any other common data files produced from the analysis of microarray based hybridization on commercially available technology platforms (see, for instance, the Affymetrix GeneChip® Expression Analysis Technical Manual available at www.affymetrix.com). Such files may or may not be annotated with various information, for instance, but not limited to, information related to the customer or remote user, cell or tissue sample data or information, hybridization technology or platform on which the data was generated and/or test agent data or information.

Once data is received, the nucleic acid hybridization data may be screened for database compatibility by any available means. In one embodiment, commonly available data quality control metrics can be applied. For instance, outlier analysis methods or techniques may be utilized to identify samples incompatible with the database, for instance, samples exhibiting erroneous florescence values from control probes which are common between the data and the database or toxicity model. In addition, various data QC metrics can be applied, including one or more disclosed in PCT/US03/24160, filed Aug. 1, 2003, which claims priority to U.S. provisional application 60/399,727.

Cell or Tissue Sample Preparation

As described above, the cell population that is exposed to the test agent, compound or composition may be exposed in vitro or in vivo. For instance, cultured or freshly isolated liver cells, in particular rat hepatocytes, may be exposed to the agent under standard laboratory and cell culture conditions. In another assay format, in vivo exposure may be accomplished by administration of the agent to a living animal, for instance a laboratory rat.

Procedures for designing and conducting toxicity tests in in vitro and in vivo systems are well known, and are described in many texts on the subject, such as Loomis et al., Loomis's Esstentials of Toxicology, 4th Ed., Academic Press, New York, 1996; Echobichon, The Basics of Toxicity Testing, CRC Press, Boca Raton, 1992; Frazier, editor, In Vitro Toxicity Testing, Marcel Dekker, New York, 1992; and the like.

In in vitro toxicity testing, two groups of test organisms are usually employed. One group serves as a control, and the other group receives the test compound in a single dose (for acute toxicity tests) or a regimen of doses (for prolonged or chronic toxicity tests). Because, in some cases, the extraction of tissue as called for in the methods of the invention requires sacrificing the test animal, both the control group and the group receiving compound must be large enough to permit removal of animals for sampling tissues, if it is desired to observe the dynamics of gene expression through the duration of an experiment.

In setting up a toxicity study, extensive guidance is provided in the literature for selecting the appropriate test organism for the compound being tested, route of administration, dose ranges, and the like. Water or physiological saline (0.9% NaCl in water) is the solute of choice for the test compound since these solvents permit administration by a variety of routes. When this is not possible because of solubility limitations, vegetable oils such as corn oil or organic solvents such as propylene glycol may be used.

Regardless of the route of administration, the volume required to administer a given dose is limited by the size of the animal that is used. It is desirable to keep the volume of each dose uniform within and between groups of animals. When rats or mice are used, the volume administered by the oral route generally should not exceed about 0.005 ml per gram of animal. Even when aqueous or physiological saline solutions are used for parenteral injection the volumes that are tolerated are limited, although such solutions are ordinarily thought of as being innocuous. The intravenous LD₅₀ of distilled water in the mouse is approximately 0.044 ml per gram and that of isotonic saline is 0.068 ml per gram of mouse. In some instances, the route of administration to the test animal should be the same as, or as similar as possible to, the route of administration of the compound to man for therapeutic purposes.

When a compound is to be administered by inhalation, special techniques for generating test atmospheres are necessary. The methods usually involve aerosolization or nebulization of fluids containing the compound. If the agent to be tested is a fluid that has an appreciable vapor pressure, it may be administered by passing air through the solution under controlled temperature conditions. Under these conditions, dose is estimated from the volume of air inhaled per unit time, the temperature of the solution, and the vapor pressure of the agent involved. Gases are metered from reservoirs. When particles of a solution are to be administered, unless the particle size is less than about 2 μm the particles will not reach the terminal alveolar sacs in the lungs. A variety of apparati and chambers are available to perform studies for detecting effects of irritant or other toxic endpoints when they are administered by inhalation. The preferred method of administering an agent to animals is via the oral route, either by intubation or by incorporating the agent in the feed.

When the agent is exposed to cells in vitro or in cell culture, the cell population to be exposed to the agent may be divided into two or more subpopulations, for instance, by dividing the population into two or more identical aliquots. In some preferred embodiments of the methods of the invention, the cells to be exposed to the agent are derived from liver tissue. For instance, cultured or freshly isolated rat hepatocytes may be used.

The methods of the invention may be used generally to predict at least one toxic response, and, as described in the Examples, may be used to predict the likelihood that a compound or test agent will induce various specific pathologies, such as liver cholestasis, genotoxicity/carcinogenesis, hepatitis, human-specific toxicity, induction of liver enlargement, steatosis, macrovesicular steatosis, microvesicular steatosis, necrosis, non-genotoxic/non-carcinogenic toxicity, peroxisome proliferation, rat non-genotoxic toxicity, general hepatotoxicity, or other pathologies associated with at least one known toxin. The methods of the invention may also be used to determine the similarity of a toxic response to one or more individual compounds. In addition, the methods of the invention may be used to predict or elucidate the potential cellular pathways influenced, induced or modulated by the compound or test agent.

Databases and Computer Systems

Databases and computer systems of the present invention typically comprise one or more data structures comprising toxicity or toxicology models as described herein, including models comprising individual gene or toxicology marker weighted index scores or PLS scores (See Table 2), gene regulation scores, sample prediction scores and/or toxicity reference prediction scores. Such databases and computer systems may also comprise software that allows a user to manipulate the database content or to calculate or generate scores as described herein, including individual gene regulation scores and sample prediction scores from nucleic acid hybridization data. The software may also allow the user to compare one or more sample prediction scores to one or more toxicity reference paradigm scores in at least one toxicity model.

As discussed above, the databases and computer systems of the invention may comprise equipment and software that allow access directly or through a remote link, such as direct dial-up access or access via a password protected Internet link.

Any available hardware may be used to create computer systems of the invention. Any appropriate computer platform, user interface, etc. may be used to perform the necessary comparisons between sequence information, gene or toxicology marker information and any other information in the database or information provided as an input. For example, a large number of computer workstations are available from a variety of manufacturers. Client/server environments, database servers and networks are also widely available and appropriate platforms for the databases of the invention.

The databases may be designed to include different parts, for instance a sequence database and a toxicology reference database. Methods for the configuration and construction of such databases and computer-readable media containing such databases are widely available, for instance, see U.S. Publication No. 2003/0171876 (Ser. No. 10/090,144), filed Mar. 5, 2002, PCT Publication No. WO 02/095659, published Nov. 23, 2002, and U.S. Pat. No. 5,953,727, which are herein incorporated by reference in their entirety. In a preferred embodiment, the database is a ToxExpress® or BioExpress™ database marketed by Gene Logic Inc., Gaithersburg, Md.

The databases of the invention may be linked to an outside or external database such as GenBank (www.ncbi.num.nih.gov/entrez.index.html); KEGG (www.genome.ad.jp/kegg); SPAD (www.grt.kyushu-u.ac.jp/spad/index.html); HUGO (www.gene.ucl.ac.uk/hugo); Swiss-Prot (www.expasy.ch.sprot); Prosite (www.expasy.ch/tools/scnpsit1.html); OMIM (www.ncbi.nlm.nih.gov/omim); and GDB (www.gdb.org). In a preferred embodiment, the external database is GenBank and the associated databases maintained by the National Center for Biotechnology Information (NCBI) (www.ncbi.nlm.nih.gov).

Any appropriate computer platform, user interface, etc. may be used to perform the necessary comparisons between sequence information, gene expression information and any other information in the database or information provided as an input. For example, a large number of computer workstations are available from a variety of manufacturers, such has those available from Silicon Graphics. Client/server environments, database servers and networks are also widely available and appropriate platforms for the databases of the invention.

The databases of the invention may be used to produce, among other things, eNortherns™ reports (Gene Logic, Inc) that allow the user to determine the cell type or tissue in which a given gene is expressed and to allow determination of the abundance or expression level of a given gene in a particular tissue or cell.

Toxicity or Toxicology Reports

As descried above, the methods, databases and computer systems of the invention can be used to produce, deliver and/or send a toxicity, hepatotoxicity or toxicology report. As consistent with the use of the terms “toxicity” and “toxicology” as used herein, a “toxicity report” and a “toxicology report” are interchangeable.

The toxicity report of the invention typically comprises information or data related to the results of the practice of a method of the invention. For instance, the practice of a method of identifying at least one toxic effect of a test agent or compound as herein described may result in the preparation or production of a report describing the results of the method. The report may comprise information related to the toxic effects predicted by the comparison of at least one sample prediction score to at least one toxicity reference prediction score from the database. The report may also present information concerning the nucleic acid hybridization data, such as the integrity of the data as well as information inputted by the user of the database and methods of the invention, such as information used to annotate the nucleic acid hybridization data.

As an exemplary, non-limiting example, a toxicity report of the invention may be in a form such as the reports disclosed in PCT/US02/22701, filed Jul. 18, 2002, which is herein incorporated by reference in its entirety. As described elsewhere in this specification, the report may be generated by a server or computer system to which is loaded nucleic acid hybridization data by a user. The report related to that nucleic acid data may be generated and delivered to the user via remote means such as a password secured environment available over the internet or via available computer communication means such as email.

Generating Nucleic Acid Hybridization Data

Any assay format to detect gene expression may be used to produce nucleic acid hybridization data. For example, traditional Northern blotting, dot or slot blot, nuclease protection, primer directed amplification, RT-PCR, semi- or quantitative PCR, branched-chain DNA and differential display methods may be used for detecting gene expression levels or producing nucleic acid hybridization data. Those methods are useful for some embodiments of the invention. In cases where smaller numbers of genes are detected, amplification based assays may be most efficient. Methods and assays of the invention, however, may be most efficiently designed with high-throughput hybridization-based methods for detecting the expression of a large number of genes.

To produce nucleic acid hybridization data, any hybridization assay format may be used, including solution-based and solid support-based assay formats. Solid supports containing oligonucleotide probes for differentially expressed genes of the invention can be filters, polyvinyl chloride dishes, particles, beads, microparticles or silicon or glass based chips, etc. Such chips, wafers and hybridization methods are widely available, for example, those disclosed by Beattie (WO 95/11755).

Any solid surface to which oligonucleotides can be bound, either directly or indirectly, either covalently or non-covalently, can be used. A preferred solid support is a high density array or DNA chip. These contain a particular oligonucleotide probe in a predetermined location on the array. Each predetermined location may contain more than one molecule of the probe, but each molecule within the predetermined location has an identical sequence. Such predetermined locations are termed features. There may be, for example, from 2, 10, 100, 1000 to 10,000, 100,000 or 400,000 or more of such features on a single solid support. The solid support, or the area within which the probes are attached may be on the order of about a square centimeter. Probes corresponding to the genes or gene fragments of Table 2 may be attached to single or multiple solid support structures, e.g., the probes may be attached to a single chip or to multiple chips to comprise a chip set. The genes or gene fragments described in the related applications mentioned above may also be attached to these solid supports.

Oligonucleotide probe arrays for expression monitoring can be made and used according to any techniques known in the art (see for example, Lockhart et al. (1996), Nat Biotechnol 14:1675-1680; McGall et al. (1996), Proc Nat Acad Sci USA 93: 13555-13460). Such probe arrays may contain at least two or more oligonucleotides that are complementary to or hybridize to two or more of the genes or gene fragments described in Table 2. For instance, such arrays may contain oligonucleotides that are complementary to or hybridize to at least about 2, 3, 4, 5, 6, 7, 8, 9, 10, 20, 30, 50, 70, 100, 500 or 1,000 or more of the genes described herein. Preferred arrays contain all, or substantially all, of the genes or gene fragments listed in Table 2. As used herein, “substantially all” of the genes in Table 2 refers to a set of genes or gene fragments containing at least 80% of the genes or gene fragments in Table 2. In another preferred embodiment, arrays are constructed that contain oligonucleotides to detect all or nearly all of the genes in Table 2 on a single solid support substrate, such as a chip.

The sequences of the genes and gene fragments of Table 2 are in the public databases. Table 1 provides the SEQ ID NO: and GenBank Accession Number (NCBI RefSeq ID) for each of the sequences (see www.ncbi.nlm.nih.gov/), as well as the title for the cluster of which gene is part. The sequences of the genes in GenBank are expressly herein incorporated by reference in their entirety as of the filing date of this application, as are related sequences, for instance, sequences from the same gene of different lengths, variant sequences, polymorphic sequences, genomic sequences of the genes and related sequences from different species, including the human counterparts, where appropriate.

In addition to gene sequences and fragments, Table 1 also provides protein sequences that correspond to the gene sequences. These proteins can be secreted or non-secreted proteins. Their annotation is based on the databases the proteins are presented. For example, if a protein fragment is linked to a RefSeq nucleotide record, the corresponding RefSeq protein record is used. If a protein fragment is linked to a Swiss-Prot protein record by Gene Index database, this Swiss-Prot protein record is used. A protein fragment could be linked to a Unigene cluster, which may have a list of annotated protein IDs. These protein IDs are produced by NCBI based on the similarity of search between each nucleotide sequence within a Unigene cluster and known protein sequences in GenBank and they may contain paralogous proteins in stead of orthologous ones. Therefore, only those that pass the cutoff score (the sequence percent identity >98% and the alignment sequence length>200) will be incorporated into the final result.

Certain genes in the commercial database may contain function annotations, which can be used to determine whether they encode secreted proteins. However, the proteins themselves may not have been identified yet. For this reason, in the protein sequence annotation in Table 1, only known protein sequence records are considered and the in-silicon translation from nucleotide sequences to protein sequences is not included.

Annotation for secreted protein sequences are conducted in several databases. Annotation from the section of “sub-cellular location” in Swiss-Prot database is collected. The following keywords are searched against the text files of the database: “secret,” “extracellular,” “hormone,” “lactation,” “milk,” “bile acid catabolism,” “cytokine,” “IgA-binding protein,” “palmitate,” “pancreas,” “pituitary,” “seminal vesicle,” “thymus,” “Wnt signaling pathway.” All positive results are annotated as secreted proteins for further analysis. In another embodiment, annotation of cellular component and molecular function from Gene Ontology is collected. Annotation for Biological process is ignored because its information is related to a set of genes instead of the gene of interest and thus causes too many false positives. Gene name and RefSeq function summary are collected. The description from InterPro protein family database is also collected. All the members of cytokine gene family (Gene Logic Gene Index database) are flagged as secreted proteins.

For each record which is annotated as a secreted protein, its description is checked to ensure false positives are removed. For example, GO:000487 is described in the database as having receptor activity when it combines with an extracellular or intracellular messenger to initiate a change in cell activity. Such a characterization would make it a false positive because it is not the protein itself but the combination of the protein and a messenger protein that may make the protein complex behave as a secreted protein. Sometimes, in order to further confirm the result, external database such as NCBI PubMed or even Google is queried.

As described above, in addition to the sequences of the GenBank Accession Numbers disclosed in the Table 2, sequences such as naturally occurring variant or polymorphic sequences may be used in the methods and compositions of the invention. For instance, expression levels of various allelic or homologous forms of a gene or gene fragment disclosed in Table 2 may be assayed. Any and all nucleotide variations that do not alter the functional activity of a gene or gene fragment listed in Table 2, including all naturally occurring allelic variants of the genes herein disclosed, may be used in the methods and to make the compositions (e.g., arrays) of the invention.

Probes based on the sequences of the genes described above may be prepared by any commonly available method. Oligonucleotide probes for screening or assaying a tissue or cell sample are preferably of sufficient length to specifically hybridize only to appropriate, complementary genes or transcripts. Typically the oligonucleotide probes will be at least about 10, 12, 14, 16, 18, 20 or 25 nucleotides in length. In some cases, longer probes of at least 30, 40, or 50 nucleotides will be desirable.

As used herein, oligonucleotide sequences that are complementary to one or more of the genes or gene fragments described in Table 2 refer to oligonucleotides that are capable of hybridizing under stringent conditions to at least part of the nucleotide sequences of said genes. Such hybridizable oligonucleotides will typically exhibit at least about 75% sequence identity at the nucleotide level to said genes, preferably about 80% or 85% sequence identity or more preferably about 90% or 95% or more sequence identity to said genes (see GeneChip® Expression Analysis Manual, Affymetrix, Rev. 3, which is herein incorporated by reference in its entirety).

Probe Design

One of skill in the art will appreciate that an enormous number of array designs are suitable for the practice of this invention. The high density array will typically include a number of test probes that specifically hybridize to the sequences of interest. Probes may be produced from any region of the genes or gene fragments identified in Table 2 and the attached representative sequence listing. In instances where the gene reference in the Tables is a gene fragment, probes may be designed from that sequence or from other regions of the corresponding full-length transcript that may be available in any of the sequence databases, such as those herein described. See WO 99/32660 for methods of producing probes for a given gene or genes. In addition, any available software may be used to produce specific probe sequences, including, for instance, software available from Molecular Biology Insights, Olympus Optical Co. and Biosoft International. In a preferred embodiment, the array will also include one or more control probes.

High density array chips of the invention include “test probes.” Test probes may be oligonucleotides that range from about 5 to about 500, or about 7 to about 50 nucleotides, more preferably from about 10 to about 40 nucleotides and most preferably from about 15 to about 35 nucleotides in length. In other particularly preferred embodiments, the probes are about 20 or 25 nucleotides in length. In another preferred embodiment, test probes are double or single strand DNA sequences. DNA sequences are isolated or cloned from natural sources or amplified from natural sources using native nucleic acid as templates. These probes have sequences complementary to particular subsequences of the genes whose expression they are designed to detect. Thus, the test probes are capable of specifically hybridizing to the target nucleic acid they are to detect.

In addition to test probes that bind the target nucleic acid(s) of interest, the high density array can contain a number of control probes. The control probes may fall into three categories referred to herein as 1) normalization controls; 2) expression level controls; and 3) mismatch controls.

Normalization controls are oligonucleotide or other nucleic acid probes that are complementary to labeled reference oligonucleotides or other nucleic acid sequences that are added to the nucleic acid sample to be screened. The signals obtained from the normalization controls after hybridization provide a control for variations in hybridization conditions, label intensity, “reading” efficiency and other factors that may cause the signal of a perfect hybridization to vary between arrays. In a preferred embodiment, signals (e.g., fluorescence intensity) read from all other probes in the array are divided by the signal (e.g., fluorescence intensity) from the control probes thereby normalizing the measurements.

Virtually any probe may serve as a normalization control. However, it is recognized that hybridization efficiency varies with base composition and probe length. Preferred normalization probes are selected to reflect the average length of the other probes present in the array, however, they can be selected to cover a range of lengths. The normalization control(s) can also be selected to reflect the (average) base composition of the other probes in the array, however in a preferred embodiment, only one or a few probes are used and they are selected such that they hybridize well (i.e., no secondary structure) and do not match any target-specific probes.

Expression level controls are probes that hybridize specifically with constitutively expressed genes in the biological sample. Virtually any constitutively expressed gene provides a suitable target for expression level controls. Typically expression level control probes have sequences complementary to subsequences of constitutively expressed “housekeeping genes” including, but not limited to the actin gene, the transferrin receptor gene, the GAPDH gene, and the like.

Mismatch controls may also be provided for the probes to the target genes, for expression level controls or for normalization controls. Mismatch controls are oligonucleotide probes or other nucleic acid probes identical to their corresponding test or control probes except for the presence of one or more mismatched bases. A mismatched base is a base selected so that it is not complementary to the corresponding base in the target sequence to which the probe would otherwise specifically hybridize. One or more mismatches are selected such that under appropriate hybridization conditions (e.g., stringent conditions) the test or control probe would be expected to hybridize with its target sequence, but the mismatch probe would not hybridize (or would hybridize to a significantly lesser extent) Preferred mismatch probes contain a central mismatch. Thus, for example, where a probe is a 20 mer, a corresponding mismatch probe will have the identical sequence except for a single base mismatch (e.g., substituting a G, a C or a T for an A) at any of positions 6 through 14 (the central mismatch).

Mismatch probes thus provide a control for non-specific binding or cross hybridization to a nucleic acid in the sample other than the target to which the probe is directed. For example, if the target is present the perfect match probes should be consistently brighter than the mismatch probes. In addition, if all central mismatches are present, the mismatch probes can be used to detect a mutation, for instance, a mutation of a gene or gene fragment in Table 2. The difference in intensity between the perfect match and the mismatch probe provides a good measure of the concentration of the hybridized material.

The terms “background” or “background signal intensity” refer to hybridization signals resulting from non-specific binding, or other interactions, between the labeled target nucleic acids and components of the oligonucleotide array (e.g., the oligonucleotide probes, control probes, the array substrate, etc.). Background signals may also be produced by intrinsic fluorescence of the array components themselves. A single background signal can be calculated for the entire array, or a different background signal may be calculated for each target nucleic acid. In a preferred embodiment, background is calculated as the average hybridization signal intensity for the lowest 5% to 10% of the probes in the array, or, where a different background signal is calculated for each target gene, for the lowest 5% to 10% of the probes for each gene. Of course, one of skill in the art will appreciate that where the probes to a particular gene hybridize well and thus appear to be specifically binding to a target sequence, they should not be used in a background signal calculation. Alternatively, background may be calculated as the average hybridization signal intensity produced by hybridization to probes that are not complementary to any sequence found in the sample (e.g. probes directed to nucleic acids of the opposite sense or to genes not found in the sample such as bacterial genes where the sample is mammalian nucleic acids). Background can also be calculated as the average signal intensity produced by regions of the array that lack any probes at all.

The phrase “hybridizing specifically to” or “specifically hybridizes” refers to the binding, duplexing, or hybridizing of a molecule substantially to or only to a particular nucleotide sequence or sequences under stringent conditions when that sequence is present in a complex mixture (e.g., total cellular) DNA or RNA.

As used herein a “probe” is defined as a nucleic acid, capable of binding to a target nucleic acid of complementary sequence through one or more types of chemical bonds, usually through complementary base pairing, usually through hydrogen bond formation. As used herein, a probe may include natural (i.e., A, G, U, C, or T) or modified bases (7-deazaguanosine, inosine, etc.). In addition, the bases in probes may be joined by a linkage other than a phosphodiester bond, so long as it does not interfere with hybridization. Thus, probes may be peptide nucleic acids in which the constituent bases are joined by peptide bonds rather than phosphodiester linkages.

Forming High Density Arrays

Methods of forming high density arrays of oligonucleotides with a minimal number of synthetic steps are known. The oligonucleotide analogue array can be synthesized on a single or on multiple solid substrates by a variety of methods, including, but not limited to, light-directed chemical coupling, and mechanically directed coupling (see Pirrung, U.S. Pat. No. 5,143,854).

In brief, the light-directed combinatorial synthesis of oligonucleotide arrays on a glass surface proceeds using automated phosphoramidite chemistry and chip masking techniques. In one specific implementation, a glass surface is derivatized with a silane reagent containing a functional group, e.g., a hydroxyl or amine group blocked by a photolabile protecting group. Photolysis through a photolithogaphic mask is used selectively to expose functional groups which are then ready to react with incoming 5′ photoprotected nucleoside phosphoramidites. The phosphoramidites react only with those sites which are illuminated (and thus exposed by removal of the photolabile blocking group). Thus, the phosphoramidites only add to those areas selectively exposed from the preceding step. These steps are repeated until the desired array of sequences have been synthesized on the solid surface. Combinatorial synthesis of different oligonucleotide analogues at different locations on the array is determined by the pattern of illumination during synthesis and the order of addition of coupling reagents.

In addition to the foregoing, additional methods which can be used to generate an array of oligonucleotides on a single substrate are described in PCT Publication Nos. WO 93/09668 and WO 01/23614. High density nucleic acid arrays can also be fabricated by depositing pre-made or natural nucleic acids in predetermined positions. Synthesized or natural nucleic acids are deposited on specific locations of a substrate by light directed targeting and oligonucleotide directed targeting. Another embodiment uses a dispenser that moves from region to region to deposit nucleic acids in specific spots.

Nucleic Acid Samples

Cell or tissue samples may be exposed to the test agent in vitro or in vivo. When cultured cells or tissues are used, appropriate mammalian cell extracts, such as liver extracts, may also be added with the test agent to evaluate agents that may require biotransformation to exhibit toxicity. In a preferred format, primary isolates or cultured cell lines of animal or human renal cells may be used.

The genes which are assayed according to the present invention are typically in the form of mRNA or reverse transcribed mRNA. The genes may or may not be cloned. The genes may or may not be amplified. The cloning and/or amplification do not appear to bias the representation of genes within a population. In some assays, it may be preferable, however, to use polyA+ RNA as a source, as it can be used with less processing steps.

As is apparent to one of ordinary skill in the art, nucleic acid samples used in the methods and assays of the invention may be prepared by any available method or process. Methods of isolating total mRNA are well known to those of skill in the art. For example, methods of isolation and purification of nucleic acids are described in detail in Chapter 3 of Laboratory Techniques in Biochemistry and Molecular Biology, Vol. 24, Hybridization With Nucleic Acid Probes: Theory and Nucleic Acid Probes, P. Tijssen, Ed., Elsevier Press, New York, 1993. Such samples include RNA samples, but also include cDNA synthesized from a mRNA sample isolated from a cell or tissue of interest. Such samples also include DNA amplified from the cDNA, and RNA transcribed from the amplified DNA. One of skill in the art would appreciate that it is desirable to inhibit or destroy RNase present in homogenates before homogenates are used.

Biological samples may be of any biological tissue or fluid or cells from any organism as well as cells raised in vitro, such as cell lines and tissue culture cells. Frequently the sample will be a tissue or cell sample that has been exposed to a compound, agent, drug, pharmaceutical composition, potential environmental pollutant or other composition. In some formats, the sample will be a “clinical sample” which is a sample derived from a patient. Typical clinical samples include, but are not limited to, sputum, blood, blood-cells (e.g., white cells), tissue or fine needle biopsy samples, urine, peritoneal fluid, and pleural fluid, or cells therefrom. Biological samples may also include sections of tissues, such as frozen sections or formalin fixed sections taken for histological purposes.

Hybridization

Nucleic acid hybridization simply involves contacting a probe and target nucleic acid under conditions where the probe and its complementary target can form stable hybrid duplexes through complementary base pairing. See WO 99/32660. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids. Under low stringency conditions (e.g., low temperature and/or high salt) hybrid duplexes (e.g., DNA:DNA, RNA:RNA, or RNA:DNA) will form even where the annealed sequences are not perfectly complementary. Thus, specificity of hybridization is reduced at lower stringency. Conversely, at higher stringency (e.g., higher temperature or lower salt) successful hybridization tolerates fewer mismatches. One of skill in the art will appreciate that hybridization conditions may be selected to provide any degree of stringency.

In a preferred embodiment, hybridization is performed at low stringency, in this case in 6× SSPET at 37° C. (0.005% Triton X-100), to ensure hybridization and then subsequent washes are performed at higher stringency (e.g., 1× SSPET at 37° C.) to eliminate mismatched hybrid duplexes. Successive washes may be performed at increasingly higher stringency (e.g., down to as low as 0.25× SSPET at 37° C. to 50° C.) until a desired level of hybridization specificity is obtained. Stringency can also be increased by addition of agents such as formamide. Hybridization specificity may be evaluated by comparison of hybridization to the test probes with hybridization to the various controls that can be present (e.g., expression level control, normalization control, mismatch controls, etc.).

In general, there is a tradeoff between hybridization specificity (stringency) and signal intensity. Thus, in a preferred embodiment, the wash is performed at the highest stringency that produces consistent results and that provides a signal intensity greater than approximately 10% of the background intensity. Thus, in a preferred embodiment, the hybridized array may be washed at successively higher stringency solutions and read between each wash. Analysis of the data sets thus produced will reveal a wash stringency above which the hybridization pattern is not appreciably altered and which provides adequate signal for the particular oligonucleotide probes of interest.

Signal Detection

The hybridized nucleic acids are typically detected by detecting one or more labels attached to the sample nucleic acids. The labels may be incorporated by any of a number of means well known to those of skill in the art. See WO 99/32660.

Kits

The invention further includes kits combining, in different combinations, high-density oligonucleotide arrays, reagents for use with the arrays, signal detection and array-processing instruments, toxicology databases and analysis and database management software described above. The kits may be used, for example, to predict or model the toxic response of a test compound.

The databases that may be packaged with the kits are described above. In particular, the database software and packaged information may contain the databases saved to a computer-readable medium, or transferred to a user's local server. In another format, database and software information may be provided in a remote electronic format, such as a website, the address of which may be packaged in the kit.

Databases and software designed for use with microarrays are discussed in Balaban et al., U.S. Pat. No. 6,229,911, a computer-implemented method for managing information collected from small or large numbers of microarrays, and U.S. Pat. No. 6,185,561, a computer-based method with data mining capability for collecting gene expression level data, adding additional attributes and reformatting the data to produce answers to various queries. Chee et al., U.S. Pat. No. 5,974,164, disclose a software-based method for identifying mutations in a nucleic acid sequence based on differences in probe fluorescence intensities between wild type and mutant sequences that hybridize to reference sequences.

Diagnostic Uses for the Toxicity Markers

As described above, the genes and gene expression information or portfolios of the genes with their expression information as provided in Tables 1 and 2 may be used as diagnostic markers for the prediction or identification of the physiological state of tissue or cell sample that has been exposed to a compound or to identify or predict the toxic effects of a compound or agent. For instance, a tissue sample such as a sample of peripheral blood cells or some other easily obtainable tissue sample may be assayed by any of the methods described above, and the expression levels from a gene or gene fragment of Table 2 may be compared to the expression levels found in tissues or cells exposed to the toxins described herein. These methods may result in the diagnosis of a physiological state in the cell or may be used to identify the potential toxicity of a compound, for instance a new or unknown compound or agent. The comparison of expression data, as well as available sequence or other information may be done by researcher or diagnostician or may be done with the aid of a computer and databases as described below.

In another format, the levels of a gene(s) of Table 2, its encoded protein(s), or any metabolite produced by the encoded protein may be monitored or detected in a sample, such as a bodily tissue or fluid sample to identify or diagnose a physiological state of an organism. Such samples may include any tissue or fluid sample, including urine, blood and easily obtainable cells such as peripheral lymphocytes.

Use of the Markers for Monitoring Toxicity Progression

As described above, the genes and gene expression information provided in Tables 1 and 2 may also be used as markers for the monitoring of toxicity progression, such as that found after initial exposure to a drug, drug candidate, toxin, pollutant, etc. For instance, a tissue or cell sample may be assayed by any of the methods described above, and the expression levels from a gene or gene fragment of Table 2 may be compared to the expression levels found in tissue or cells exposed to the hepatotoxins described herein. The comparison of the expression data, as well as available sequence or other information may be done by researcher or diagnostician or may be done with the aid of a computer and databases.

Use of the Toxicity Markers for Drug Screening

According to the present invention, the genes and gene fragments identified in Tables 1 and 2 may be used as markers or drug targets to evaluate the effects of a candidate drug, chemical compound or other agent on a cell or tissue sample. The genes may also be used as drug targets to screen for agents that modulate their expression and/or activity. In various formats, a candidate drug or agent can be screened for the ability to stimulate the transcription or expression of a given marker or markers or to down-regulate or counteract the transcription or expression of a marker or markers. According to the present invention, one can also compare the specificity of a drug's effects by looking at the number of markers which the drug induces and comparing them. More specific drugs will have less transcriptional targets. Similar sets of markers identified for two drugs may indicate a similarity of effects.

Assays to monitor the expression of a marker or markers as defined in Tables 1 and 2 may utilize any available means of monitoring for changes in the expression level of the nucleic acids of the invention. As used herein, an agent is said to modulate the expression of a nucleic acid of the invention if it is capable of up- or down-regulating expression of the nucleic acid in a cell.

In one assay format, gene chips containing probes to one, two or more genes or gene fragments from Table 2 may be used to directly monitor or detect changes in gene expression in the treated or exposed cell. Cell lines, tissues or other samples are first exposed to a test agent and in some instances, a known toxin, and the detected expression levels of one or more, or preferably 2 or more of the genes or gene fragments of Table 2 are compared to the expression levels of those same genes exposed to a known toxin alone. Compounds that modulate the expression patterns of the known toxin(s) would be expected to modulate potential toxic physiological effects in vivo. The genes and gene fragments in Table 2 are particularly appropriate markers in these assays as they are differentially expressed in cells upon exposure to a known hepatotoxin.

In another format, cell lines that contain reporter gene fusions between the open reading frame and/or the transcriptional regulatory regions of a gene or gene fragment in Table 2 and any assayable fusion partner may be prepared. Numerous assayable fusion partners are known and readily available including the firefly luciferase gene and the gene encoding chloramphenicol acetyltransferase (Alam et al. (1990) Anal Biochem 188:245-254). Cell lines containing the reporter gene fusions are then exposed to the agent to be tested under appropriate conditions and time. Differential expression of the reporter gene between samples exposed to the agent and control samples identifies agents which modulate the expression of the nucleic acid.

Additional assay formats may be used to monitor the ability of the agent to modulate the expression of a gene identified in Table 2. For instance, as described above, mRNA expression may be monitored directly by hybridization of probes to the nucleic acids of the invention. Cell lines are exposed to the agent to be tested under appropriate conditions and time and total RNA or mRNA is isolated by standard procedures such those disclosed in Sambrook et al. (Molecular Cloning: A Laboratory Manual, Third Ed., Cold Spring Harbor Laboratory Press, Cold Spring Harbor, N.Y., 2001).

Another embodiment of the present invention provides methods for identifying agents that modulate at least one activity of a protein(s) encoded by the genes or gene fragments in Table 2. Such methods or assays may utilize any means of monitoring or detecting the desired activity.

In one format, the relative amounts of a protein encoded by a gene of Table 2 between a cell population that has been exposed to the agent to be tested compared to an un-exposed control cell population and a cell population exposed to a known toxin may be assayed. In this format, probes such as specific antibodies are used to monitor the differential expression of the protein in the different cell populations. Cell lines or populations are exposed to the agent to be tested under appropriate conditions and time. Cellular lysates may be prepared from the exposed cell line or population and a control, unexposed cell line or population. The cellular lysates are then analyzed with the probe, such as a specific antibody.

Agents that are assayed in the above methods can be randomly selected or rationally selected or designed. As used herein, an agent is said to be randomly selected when the agent is chosen randomly without considering the specific sequences involved in the association of the a protein of the invention alone or with its associated substrates, binding partners, etc. An example of randomly selected agents is the use a chemical library or a peptide combinatorial library, or a growth broth of an organism.

As used herein, an agent is said to be rationally selected or designed when the agent is chosen on a nonrandom basis which takes into account the sequence of the target site and/or its conformation in connection with the agent's action. Agents can be rationally selected or rationally designed by utilizing the peptide sequences that make up these sites. For example, a rationally selected peptide agent can be a peptide whose amino acid sequence is identical to or a derivative of any functional consensus site.

The agents of the present invention can be, as examples, peptides, small molecules, vitamin derivatives, as well as carbohydrates. Dominant negative proteins, DNAs encoding these proteins, antibodies to these proteins, peptide fragments of these proteins or mimics of these proteins may be introduced into cells to affect function. “Mimic” used herein refers to the modification of a region or several regions of a peptide molecule to provide a structure chemically different from the parent peptide but topographically and functionally similar to the parent peptide (see G. A. Grant in: Molecular Biology and Biotechnology, Meyers, ed., pp. 659-664, VCH Publishers, New York, 1995). A skilled artisan can readily recognize that there is no limit as to the structural nature of the agents of the present invention.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### EXAMPLES

### Example 1

### Generation of Toxicity Models using RMA and PLS

Various liver toxins are administered to male Sprague-Dawley rats at various timepoints using administration diluents, protocols, dosing regimes and sampling (sacrifice) time points as previously described in the art, as previously described in the related applications discussed above, and as indicated in Table 4.

Observation of Animals

1. Clinical cage side observations- twice daily mortality and moribundity check. Skin and fur, eyes and mucous membrane, respiratory system, circulatory system, autonomic and central nervous system, somatomotor pattern, and behavior pattern are checked. Potential signs of toxicity, including tremors, convulsions, salivation, diarrhea, lethargy, coma or other atypical behavior or appearance, are recorded as they occur and include a time of onset, degree, and duration.

2. Physical Examinations-Prior to randomization, prior to initial treatment, and prior to sacrifice.

3. Body Weights-Prior to randomization, prior to initial treatment, and prior to sacrifice.

Clinical Pathology

1. Frequency- Prior to necropsy.

2. Number of animals- All surviving animals.

3. Bleeding Procedure- Blood was obtained by puncture of the orbital sinus while under 70% CO₂/30% O₂ anesthesia.

4. Collection of Blood Samples-Approximately 0.5 mL of blood is collected into EDTA tubes for evaluation of hematology parameters. Approximately 1 mL of blood is collected into serum separator tubes for clinical chemistry analysis. Approximately 200 μL of plasma is obtained and frozen at ˜−80° C. for test compound/metabolite estimation. An additional ˜2 mL of blood is collected into a 15 mL conical polypropylene vial to which ˜3 mL of Trizol is immediately added. The contents are immediately mixed with a vortex and by repeated inversion. The tubes are frozen in liquid nitrogen and stored at ˜−80° C.

Termination Procedures

Terminal Sacrifice

At the time points indicated above, rats are weighed, physically examined, sacrificed by decapitation, and exsanguinated. The animals are necropsied within approximately five minutes of sacrifice. Separate sterile, disposable instruments are used for each animal. Necropsies are conducted on each animal following procedures approved by board-certified pathologists.

Animals not surviving until terminal sacrifice are discarded without necropsy (following euthanasia by carbon dioxide asphyxiation, if moribund). The approximate time of death for moribund or found dead animals is recorded.

Postmortem Procedures

All tissues are collected and frozen within approximately 5 minutes of the animal's death. Tissues are stored at approximately −80° C. or preserved in 10% neutral buffered formalin.

Tissue Collection and Processing

Liver

1. Right medial lobe -snap freeze in liquid nitrogen and store at ˜−80° C.
2. Left medial lobe -Preserve in 10% neutral-buffered formalin (NBF) and evaluate for gross and microscopic pathology.
3. Left lateral lobe -snap freeze in liquid nitrogen and store at ˜−80° C.

Heart

1. A sagittal cross-section containing portions of the two atria and of the two ventricles is preserved in 10% NBF. The remaining heart is frozen in liquid nitrogen and stored at ˜−80° C.

Kidneys (both)

1. Left—Hemi-dissect; half is preserved in 10% NBF and the remaining half is frozen in liquid nitrogen and stored at ˜−80° C.
2. Right—Hemi-dissect; half is preserved in 10% NBF and the remaining half is frozen in liquid nitrogen and stored at ˜−80° C.

Testes (both)—A sagittal cross-section of each testis is preserved in 10% NBF. The remaining testes are frozen together in liquid nitrogen and stored at ˜−80° C.

Brain (whole)—A cross-section of the cerebral hemispheres and of the diencephalon are preserved in 10% NBF, and the rest of the brain is frozen in liquid nitrogen and stored at ˜−80° C.

Microarray sample preparation is conducted with minor modifications, following the protocols set forth in the Affymetrix GeneChip® Expression Technical Analysis Manual (Affymetrix, Inc. Santa Clara, Calif.). Frozen tissue is ground to a powder using a Spex Certiprep 6800 Freezer Mill. Total RNA is extracted with Trizol (Invitrogen, Carlsbad Calif.) utilizing the manufacturer's protocol. mRNA is isolated using the Oligotex mRNA Midi kit (Qiagen) followed by ethanol precipitation. Double stranded cDNA is generated from mRNA using the SuperScript Choice system (Invitrogen, Carlsbad Calif.). First strand cDNA synthesis is primed with a T7-(dT24) oligonucleotide. The cDNA is phenol-chloroform extracted and ethanol precipitated to a final concentration of 1 μg/ml. From 2 μg of cDNA, cRNA is synthesized using Ambion's T7 MegaScript in vitro Transcription Kit.

To biotin label the cRNA, nucleotides Bio-11-CTP and Bio-16-UTP (Enzo Diagnostics) are added to the reaction. Following a 37° C. incubation for six hours, impurities are removed from the labeled cRNA following the RNeasy Mini kit protocol (Qiagen). cRNA is fragmented (fragmentation buffer consisting of 200 mM Tris-acetate, pH 8.1, 500 mM KOAc, 150 mM MgOAc) for thirty-five minutes at 94° C. Following the Affymetrix protocol, 55 μg of fragmented cRNA is hybridized on the Affymetrix rat array set for twenty-four hours at 60 rpm in a 45° C. hybridization oven. The chips are washed and stained with Streptavidin Phycoerythrin (SAPE) (Molecular Probes) in Affymetrix fluidics stations. To amplify staining, SAPE solution is added twice with an anti-streptavidin biotinylated antibody (Vector Laboratories) staining step in between. Hybridization to the probe arrays is detected by fluorometric scanning (Hewlett Packard Gene Array Scanner). Data is analyzed using Affymetrix GeneChip® and Expression Data Mining (EDMT) software, the GeneExpress® database, and S-Plus® statistical analysis software (Insightful Corp.).

Identification of Toxicity Markers and Model Building using RMA and PLS Algorithms

RMA/PLS models are built as follows. From DNA microarray data from one or more studies, a matrix of RMA fold-change expression values is generated. These values are generated, for example, according to the method of Irizarry et al. (Nucl Acids Res 31(4):e15, 2003), which uses the following equation to produce a log scale linear additive model: T(PMᵢⱼ)=eᵢ+aⱼ+εᵢⱼ. T represents the transformation that corrects for background and normalizes and converts the PM (perfect match) intensities to a log scale. eᵢ represents the log2 scale expression values found on arrays i=1−I, aⱼ represents the log scale affinity effects for probes j=1−J, and εᵢⱼ represents error (to correct for the differences in variances when using probes that bind with different intensities).

In RMA fold-change matrices, the rows represent individual fragments, and the columns are individual samples. A vehicle cohort median matrix is then calculated, in which the rows represent fragments and the columns represent vehicle cohorts, one cohort for each study/time-point combination. The values in this matrix are the median RMA expression values across the samples within those cohorts. Next, a matrix of normalized RMA expression values is generated, in which the rows represent individual fragments and the columns are individual samples. The normalized RMA values are the RMA values minus the value from the vehicle cohort median matrix corresponding to the time-matched vehicle cohort. PLS modeling is then applied to the normalized RMA matrix (a subset by taking certain fragments as described below), using a −1=non-tox, +1=tox supervised score vector as the dependant variable and the rows of normalized RMA matrix as the independent variables. PLS works by computing a series of PLS components, where each component is a weighted linear combination of fragment values. We use the nonlinear iterative partial least squares method to compute the PLS components.

To select fragments, a vehicle cohort mean matrix is generated, in which the rows represent fragments and the columns represent vehicle cohorts, one cohort for each study/time-point combination. The values in this matrix are the mean RMA expression values across the samples within those cohorts. A treated cohort mean matrix is then generated, in which the rows represent fragments and the columns represent treated (non-vehicle) cohorts, one cohort for each study/time-point/compound/dose combination. The values in this matrix are the mean RMA expression values across the samples within those cohorts. Next, a treated cohort fold-change matrix is generated, in which the rows represent fragments and the columns represent treated cohorts, one cohort for each study/time-point/compound/dose combination. The values in this matrix are the values in the treated cohort mean matrix minus the values in the vehicle cohort mean matrix corresponding to appropriate time-matched vehicle cohorts. Subsequently, a treated cohort p-value matrix is generated, in which the rows represent fragments and the columns represent treated cohorts, one cohort for each study/time-point/compound/dose combination. The values in this matrix are p-values based on two-sample t-tests comparing the treated cohort mean values to the vehicle cohort mean values corresponding to appropriate time-matched vehicle cohorts. This matrix is converted to a binary coding based on the p-values being less than 0.05 (coded as 1) or greater than 0.05 (coded as 0).

The row sums of the binary treated cohort p-value matrix are computed, where that row sum represents a “gene regulation score” for each fragment, representing the total number of treated cohorts where the fragment showed differential regulation (up- or down-regulation) compared to its time-matched vehicle cohort. PLS modeling and ⅔/⅓ cross-validation are then performed based on taking the top N fragments according to the regulation score, varying N and the number of PLS components, and recording the model success rate for each combination. N is chosen to be the point at which the cross-validated error rate are minimized. In the PLS model, each of those N fragments receives a PLS weight (PLS score) corresponding to the fragment's utility, or predictive ability, in the model (see Table 2 for lists of PLS weight scores for individual genes and gene fragments in the various hepatotoxicity models). Table 2 presents 13 hepatotoxicity models and includes the gene or gene fragment name for each marker and the corresponding PLS weight or index score for each gene or gene fragment in each model. The models are as follows: General Toxicity, Negative Control, Pharmacologically Active-negative control, Genotoxic Carcinogen, Non-genotoxic/Non-carcinogenic, Rat-specific-non-genotoxic, Cholestasis, Hepatitis, Human-specific Toxicity, Inducer of Liver Enlargement, Peroxisome Proliferator, Microvesicular Steatosis, Macrovesicular Steatosis, General Steatosis and Necrosis.

To establish a toxicity prediction score cut-off value for a toxicity model, the true-positive and false positive rates for each possible score cut-off value are computed, using the scores from all tox and non-tox samples in the training set. This generates an ROC curve, which is used to set the cut-off score at the point on the ROC curve corresponding to ˜5% false positive rate.

| Model | Cut-off score |
|---|---|
| general | 0.256 |
| cholestasis | 0.146 |
| genotoxictiy/carcinogenesis | 0.197 |
| hepatitis | 0.26 |
| human-specific toxicity | 0.28 |
| inducer of liver enlargement | 0.253 |
| macrovesicular steatosis | 0.168 |
| microvesicular steatosis | 0.227 |
| necrosis | 0.295 |
| non-genotoxicity/non- | 0.166 |
| carcinogenesis | |
| peroxisome proliferation | 0.095 |
| rat non-genotoxicity | 0.147 |
| steatosis | 0.232 |

The model can be trained by setting a score of −1 for each gene that cannot predict a toxic response and by setting a score of +1 for each gene that can predict a toxic response. Cross-validation of RMA/PLS models may be performed by the compound-drop method and by the ⅔:⅓ method. In the compound-drop method, sample data from animals treated with one particular test compound are removed from a model, and the ability of this model to predict toxicity is compared to that of a model containing a full data set. In the ⅔:⅓ method, gene expression information from a random third of the genes in the model is removed, and the ability of this subset model to predict toxicity is compared to that of a model containing a full data set.

### Example 2

### Methods of Predicting at Least One Toxic Effect of a Test Agent

To determine whether or not a sample from an animal treated with a test agent or compound exhibits at least one toxic effect or response, RNA is prepared from a cell or tissue sample exposed to the agent and hybridized to a DNA microarray, as described in Example 1 above. From the nucleic acid hybridization data, a prediction score is calculated for that sample and compared to a reference score from a toxicity reference database according to the following equation. The sample prediction score=Σwᵢ R^{FC}^{ᵢ}. “i” is the index number for each gene in a gene expression profile to be evaluated. “wᵢ” is the PLS weight score (or PLS index score, see Table 2 for the lists of PLS scores for each hepatotoxicity model) for each gene. “R^{FC}^{ᵢ}” is the RMA fold-change value for the i^{th} gene, as determined from a normalized RMA matrix of gene expression data from the sample (described above). The PLS weight multiplied by the RMA fold-change value gives a gene regulation score for each gene, and the regulation scores for all the individual genes are added to give a prediction score for the sample.

As a quality control (QC) check, for each incoming study, an average correlation assessment may be performed. After the RMA matrix is generated (genes by samples), a Pearson correlation matrix is calculated of the samples to each other. This matrix is samples by samples. For each sample row of the matrix, the mean of all correlation values in that row of the matrix, excluding the diagonal is calculated (which is always 1). This mean is the average correlation for that sample. If the average correlation is less than a threshold (for instance 0.90), the sample is flagged as a potential outlier. This process is repeated for each row (sample) in the study. Outliers flagged by the average correlation QC check are dropped out of any downstream normalization, prediction or compound similarity steps in the process.

In the liver toxicity models of Table 2, the cut-off prediction scores range from about 0.095 to about 0.295, as indicated below. If a sample score, when compared to a particular liver toxicity model, e.g. the cholestasis pathology model, is about 0.146 or above, it can be predicted that the sample shows a toxic response after exposure to the test compound. If the sample score is below 0.146, it can be predicted that the sample does not show a toxic response.

Compound similarity is assessed in the following way. In the same manner as described above, a cohort fold change vector for each study/time-point/compound/dose combination is calculated. This vector is reduced to only the fragments used in the PLS predictive models. We then calculate Pearson correlations of that cohort fold change vector to each subsetted cohort vector in our reference database. Finally, Pearson correlations are calculated ranked from highest to lowest and the results are stored in the toxicity model and reported.

A report may be generated comprising information or data related to the results of the methods of predicting at least one toxic effect. The report may comprise information related to the toxic effects predicted by the comparison of at least one sample prediction score to at least one toxicity reference prediction score from the database. The report may also present information concerning the nucleic acid hybridization data, such as the integrity of the data as well as information inputted by the user of the database and methods of the invention, such as information used to annotate the nucleic acid hybridization data. See PCT US02/22701 for a non-limiting example of a toxicity report that may be generated.

Although the present invention has been described in detail with reference to examples above, it is understood that various modifications can be made without departing from the spirit of the invention. Accordingly, the invention is limited only by the following claims. All cited patents, patent applications and publications referred to in this application are herein incorporated by reference in their entirety.

Lengthy table referenced here
US20110071767A1-20110324-T00001
Please refer to the end of the specification for access instructions.

Lengthy table referenced here
US20110071767A1-20110324-T00002
Please refer to the end of the specification for access instructions.

Lengthy table referenced here
US20110071767A1-20110324-T00003
Please refer to the end of the specification for access instructions.

Lengthy table referenced here
US20110071767A1-20110324-T00004
Please refer to the end of the specification for access instructions.

Lengthy table referenced here
US20110071767A1-20110324-T00005
Please refer to the end of the specification for access instructions.

Lengthy table referenced here
US20110071767A1-20110324-T00006
Please refer to the end of the specification for access instructions.

LENGTHY TABLES
The patent application contains a lengthy table section. A copy of the table is available in electronic form from the USPTO web site (http://seqdata.uspto.gov/?pageRequest=docDetail&DocID=US20110071767A1). An electronic copy of the table will also be available from the USPTO upon request and payment of the fee set forth in 37 CFR 1.19(b)(3).

1. A method of predicting at least one toxic effect of a test agent comprising:
(a) providing nucleic acid hybridization data for a plurality of genes from at least one liver cell or tissue sample exposed to the test agent;
(b) converting the hybridization data from at least one gene corresponding to a gene or gene fragment of Table 2 to a gene expression measure;
(c) generating a gene regulation score from the gene expression measure for said at least one gene;
(d) generating a sample prediction score for the agent; and
(e) comparing the sample prediction score to a liver toxicity reference prediction score, thereby predicting at least one toxic effect of the test agent.

2. A method of claim 1, wherein at least one cell or tissue sample is exposed to a test agent vehicle.

3. A method of claim 2, wherein step (b) comprises normalizing the hybridization data for background hybridization and for test agent vehicle induced expression.

4. A method of claim 2, wherein the gene expression measure is a gene fold change value.

5. A method of claim 4, wherein the fold change value is calculated by a log scale linear additive model.

6. A method of claim 5, wherein the log scale linear additive model is a robust multi-array (RMA).

7. A method of claim 1, wherein the nucleic acid hybridization data has been screened by a quality control process that measures outlier data.

8. A method of claim 1, wherein step (c) comprises dimension reduction using Partial Least Squares (PLS).

9. A method of claim 1, wherein the sample prediction score is generated with a weighted index score for each gene.

10. A method of claim 9, wherein the weighted index score is a PLS score from Table 2.

11. A method of claim 1, wherein the sample prediction score for the agent is generated from the gene regulation score for said at least one gene.

12. A method of claim 11, wherein the sample prediction score for the agent is generated from the gene regulation score for at least about 10 genes.

13. A method of claim 11, wherein the sample prediction score for the agent is generated from the gene regulation score for at least about 50 genes.

14. A method of claim 11, wherein the sample prediction score for the agent is generated from the gene regulation score for at least about 100 genes.

15. A method of claim 1, wherein the toxicity reference prediction score is generated by a method comprising:
(a) providing nucleic acid hybridization data for a plurality of genes from at least one liver cell or tissue sample exposed to a hepatotoxin and at least one liver cell or tissue sample exposed to the toxin vehicle;
(b) converting the hybridization data from at least one gene to fold change values;
(c) generating a gene regulation score from the fold change value for said at least one gene; and
(d) generating a toxicity reference prediction score for the toxin.

16. A method of claim 15, wherein the hepatotoxin is selected from the group consisting of: 17-alpha-ethynylestradiol (EE), 2-acetylaminofluorene (2 -AAF), 3-methylcholanthrene, Abacavir, acetaminophen (APAP; paracetamol), acetylsalicylic acid (aspirin), allyl alcohol, Amineptine, Amiodarone, Amitriptyline, ANIT (1-naphthyl isothiocyanate), Eisai (Aricept™), Aroclor 1254, AY-25329, B1 compound, Bicalutamide, bromobenzene, Bupropion, Carbamazepine, carbon tetrachloride (CCl4), Ceftazidime, chloroform, Chlorpheniramine maleate (ChlorTrimeton), CI 1000, Ciprofibrate, Clofibrate, Colchicine, Compound A (AZ), Compound B (AZ), Sulindac, Cyproterone acetate, CZB777, Dantrolene, Demeclocycline, Diclofenac, diethylnitrosamine (DEN), Diflunisal, Diphenhydramine, Diquat, DMN (dimethylnitrosamine), dopamine, Epirubicin, Erythromycin estolate, ethanol, Etoposide, Famotidine (Pepcid AC), Felbamate, Fenofibrate, Flutamide, Gemfibrozil, Gentamicin, Hydrazine (Isoniazid), hydroxyurea, Indomethacin, Labetalol, L-ethionine, LPS (lipopolysaccharide), mannitol (d-mannitol) Menadione, Metformin, Methapyrilene, Methotrexate, methyldopa, Lovastatin (Mevacor), Monocrotaline, Org 10000, Org 20000, Paraquat, Perhexilene, Phenacetin, phenobarbital, physostigmine, Plicamycin, Rifabutin, Rifampin, Rosiglitazone, Simvastatin, Stavudine, Streptomycin, Tacrine, Tamoxifen, TCDD (2,3,7,8-tetrachlorodibenzo-p-dioxin), Temozolomide, Tetracycline, thioacetamide, Valproate, Wy-14,643, Zidovudine and Zileuton.

17. A method of claim 1, wherein step (a) comprises loading nucleic acid hybridization data to a server via a remote connection.

18. A method of claim 17, wherein the remote connection is over the internet.

19. A method of claim 1, wherein the toxicity reference prediction score is provided in a database.

20. A method of claim 19, wherein the toxicity reference prediction score is derived from a hepatotoxicity model.

21. A method of claim 20, wherein the toxicity model is selected from the group consisting of an individual toxin model, a general toxicity model and a tissue pathology model.

22. A method of claim 21, wherein the general toxicity model is a general hepatotoxicity model.

23. A method of claim 21, wherein the toxicity model is a human-specific hepatotoxicity model.

24. A method of claim 21, wherein the tissue pathology model is selected from the group consisting of: a general toxicity model, a negative control model, a pharmacologically active, negative control model, a genotoxic/carcinogenic model, a non-genotoxic model, a rat-specific, non-genotoxic model, a cholestasis model, a hepatitis model, a human-specific toxicity model, an inducer of liver enlargement model, a peroxisome proliferator model, a microvesicular steatosis model, a macrovesicular steatosis model, a general steatosis model and a necrosis model.

25. A method of claim 1, further comprising: (f) generating a report comprising information related to the toxic effect.

26. A method of claim 25, wherein the report comprises information related to the mechanism of the toxic effect.

27. A method of claim 25, wherein the report comprises information related to the toxins used to prepare the toxicity reference prediction score.

28. A method of 25, wherein the report comprises information related to at least one similarity between the test agent and a toxin.

29. A method of claim 18, wherein the hybridization data is contained in a plain text file.

30. A method of claim 18, wherein the hybridization data is contained in a CEL file.

31. A method of claim 1, wherein the nucleic acid hybridization data is annotated with information selected from the group consisting of customer data, cell or tissue sample data, hybridization technology data and test agent data.

32. A method of claim 17, wherein step (a) further comprises selecting at least one toxicity model to predict said at least one toxic effect.

33. A method of providing a report comprising a prediction of at least one toxic effect of a test agent comprising:
(a) receiving nucleic acid hybridization data for a plurality of genes from at least one liver cell or tissue sample exposed to the test agent and at least one liver cell or tissue sample exposed to the test agent vehicle to a server via a remote link, wherein said plurality of genes is selected from the genes or gene fragments of Table 2;
(b) converting the hybridization data from at least one gene to robust multi-array (RMA) fold change values;
(c) generating a gene regulation score from the RMA fold change value for said at least one gene;
(d) generating a sample prediction score for the agent;
(e) comparing the sample prediction score to a toxicity reference prediction score; and
(f) providing a report comprising information related to said at least one toxic effect.

34. A method of creating a toxicity model comprising:
(a) providing nucleic acid hybridization data for a plurality of genes from at least one liver cell or tissue sample exposed to a toxin;
(b) converting the hybridization data from at least one gene corresponding to a gene or gene fragment of Table 2 to a gene expression measure;
(c) generating a gene regulation score from gene expression measure for said at least one gene;
(d) generating a toxicity reference prediction score for the toxin, thereby creating a toxicity model.

35. A method of claim 34, wherein the toxin is selected from the group consisting of: 17-alpha-ethynylestradiol (EE), 2-acetylaminofluorene (2-AAF), 3-methylcholanthrene, Abacavir, acetaminophen (APAP; paracetamol), acetylsalicylic acid (aspirin), allyl alcohol, Amineptine, Amiodarone, Amitriptyline, ANIT (1-naphthyl isothiocyanate), Eisai (Aricept™), Aroclor 1254, AY-25329, BI compound, Bicalutamide, bromobenzene, Bupropion, Carbamazepine, carbon tetrachloride (CCl4), Ceftazidime, chloroform, Chlorpheniramine maleate (ChlorTrimeton), CI 1000, Cipro fibrate, Clofibrate, Colchicine, Compound A (AZ), Compound B (AZ), Sulindac, Cyproterone acetate, CZB777, Dantrolene, Demeclocycline, Diclofenac, diethylnitrosamine (DEN), Diflunisal, Diphenhydramine, Diquat, DMN (dimethyhiitrosamine), dopamine, Epirubicin, Erythromycin estolate, ethanol, Etoposide, Famotidine (Pepcid AC), Felbamate, Fenofibrate, Flutamide, Gemfibrozil, Gentamicin, Hydrazine (Isoniazid), hydroxyurea, Indomethacin, Labetalol, L-ethionine, LPS (lipopolysaccharide), mannitol (d-mannitol) Menadione, Metformin, Methapyrilene, Methotrexate, methyldopa, Lovastatin (Mevacor), Monocrotaline, Org 10000, Org 20000, Paraquat, Perhexilene, Phenacetin, phenobarbital, physostigmine, Plicamycin, Rifabutin, Rifampin, Rosiglitazone, Simvastatin, Stavudine, Streptomycin, Tacrine, Tamoxifen, TCDD (2,3,7,8-tetrachlorodibenzo-p-dioxin), Temozolomide, Tetracycline, thioacetamide, Valproate, Wy-14,643, Zidovudine and Zileuton.

36. A method of claim 34, wherein at least one cell or tissue sample is exposed to a test agent vehicle.

37. A method of claim 34, wherein the step (b) comprises normalizing the hybridization data for background hybridization and for test agent vehicle induced expression.

38. A method of claim 34, wherein the gene expression measure is a gene fold change value.

39. A method of claim 34, wherein the fold change value is calculated by a log scale linear additive model.

40. A method of claim 34, wherein the log scale linear additive model is a robust multi-array (RMA).

41. A method of claim 34, wherein the generating of step (c) comprises dimension reduction using Partial Least Squares (PLS).

42. A method of claim 34, wherein step (d) comprises the generation of a weighted index score for each gene.

43. A method of claim 34, wherein the toxicity reference prediction score for the toxin is generated from the gene regulation score for said at least one gene.

44. A method of claim 43, wherein the toxicity reference prediction score for the agent is generated from the gene regulation score for at least about 10 genes.

45. A method of claim 43, wherein the toxicity reference prediction score for the agent is generated from the gene regulation score for at least about 50 genes.

46. A method of claim 43, wherein the toxicity reference prediction score for the agent is generated from the gene regulation score for at least about 100 genes.

47. A method of claim 34, wherein the toxicity model is selected from the group consisting of an individual toxin model, a general toxicity model and a tissue pathology model.

48. A method of claim 47, wherein the general toxicity model is a general hepatotoxicity model.

49. A method of claim 47, wherein the toxicity model is a human-specific hepatotoxicity model.

50. A method of claim 47, wherein the tissue pathology model is selected from the group consisting of: a general toxicity model, a negative control model, a pharmacologically active, negative control model, a genotoxic/carcino genie model, a non-genotoxic model, a rat-specific, non-genotoxic model, a cholestasis model, a hepatitis model, a human-specific toxicity model, an inducer of liver enlargement model, a peroxisome proliferator model, a microvesicular steatosis model, a macrovesicular steatosis model, a general steatosis model and a necrosis model.

51. A method of claim 34, further comprising validating the model.

52. A method of claim 51, wherein the validation comprises using a cross-validation procedure.

53. A method of claim 52, wherein the cross-validation procedure is a ⅔/⅓ validation procedure.

54. A computer system comprising:
(a) a computer readable medium comprising a toxicity model for predicting toxicity of a test agent, wherein the toxicity model is generated by a method of claim 29; and
(b) software that allows a user to predict at least one toxic effect of a test agent by comparing a sample prediction score to a toxicity reference prediction score in the toxicity model.

55. A computer system of claim 54, wherein the toxicity model comprises a model selected from Table 2.

56. A computer system of claim 54, wherein the toxicity model comprises weighted index scores for at least one gene or gene fragment of Table 2.

57. A computer system of claim 56, wherein the toxicity model comprises weighted index scores for at least 50 genes or gene fragments of Table 2.

58. A computer system of claim 56, wherein the toxicity model comprises weighted index scores for at least 100 genes or gene fragments of Table 2.

59. A computer system of claim 56, wherein the toxicity model comprises weighted index scores for nearly all the genes or gene fragments of Table 2.

60. A computer system of claim 54, wherein the software allows a user to calculate a sample prediction score from the nucleic acid hybridization data.

61. A computer system of claim 54, wherein the software enables a user to compare quantitative gene expression information obtained from a cell or tissue sample exposed to a test agent to the quantitative gene expression information in the toxicity model to predict whether the test agent is a toxin.

62. A computer system of claim 54, further comprising software that allows a user to transmit from a remote location nucleic acid hybridization data from a cell or tissue sample exposed to a test agent to predict whether the test agent is a toxin.

63. A computer system of claim 54, wherein the nucleic acid hybridization data from the sample may be transmitted via the Internet.

64. A computer system of claim 54, wherein the nucleic acid hybridization data is microarray hybridization data.

65. A computer system of claim 54, wherein the nucleic acid hybridization data is PCR data.

66. A computer system of claim 54, further comprising a data structure comprising at least one toxicity reference prediction score.

67. A computer system of claim 54, wherein the data structure further comprises at least one gene PLS score.

68. A computer system of claim 54, wherein the data structure further comprises at least one gene regulation score.

69. A computer system of claim 54, wherein the data structure further comprises at least one sample prediction score.

70. A computer readable medium comprising a data structure comprising at lest least one toxicity reference prediction score and software for accessing said data structure.

71. A computer system of claim 54, wherein the toxicity model is selected from the group consisting of an individual toxin model, a general toxicity model and a tissue pathology model.

72. A computer system of claim 54, wherein the general toxicity model is a general hepatotoxicity model.

73. A computer system of claim 54, wherein the toxicity model is a human-specific hepatotoxicity model.

74. A computer system of claim 54, wherein the tissue pathology model is selected from the group consisting of: a general toxicity model, a negative control model, a pharmacologically active, negative control model, a genotoxic/carcinogenic model, a non-genotoxic model, a rat-specific, non-genotoxic model, a cholestasis model, a hepatitis model, a human-specific toxicity model, an inducer of liver enlargement model, a peroxisome proliferator model, a microvesicular steatosis model, a macrovesicular steatosis model, a general steatosis model and a necrosis model.

75. A solid support comprising at least two probes, wherein each of the probes comprises a sequence that specifically hybridizes to a gene or gene fragment in Table 2.

76. A solid support of claim 75, wherein each of the probes comprises a sequence that specifically hybridizes to at least 5 genes or gene fragments in Table 2.

77. A solid support of claim 75, wherein each of the probes comprises a sequence that specifically hybridizes to at least 10 genes or gene fragments in Table 2.

78. A solid support of claim 75, wherein each of the probes comprises a sequence that specifically hybridizes to at least 50 genes or gene fragments in Table 2.

79. A solid support of claim 75, wherein each of the probes comprises a sequence that specifically hybridizes to at least 100 genes or gene fragments in Table 2.

80. A solid support of claim 75, wherein the solid support is an array comprising probes which individually specifically hybridize to substantially all of the genes or gene fragments in Table 2.

81. A solid support of claim 75, wherein the solid support is selected from the group consisting of a membrane, a glass support, a collection of beads and a silicon support.

82. A solid support of claim 75, wherein the solid support is an array comprising at least 10 different oligonucleotides in discrete locations per square centimeter.

83. A solid support of claim 82, wherein the array comprises at least about 100 different oligonucleotides in discrete locations per square centimeter.

84. A solid support of claim 82, wherein the array comprises at least about 1000 different oligonucleotides in discrete locations per square centimeter.

85. A solid support of claim 82, wherein the array comprises at least about 10,000 different oligonucleotides in discrete locations per square centimeter.

86. A method of predicting at least one toxic effect of a compound, comprising:
(a) detecting the level of expression in a tissue or cell sample exposed to the compound often or more genes or proteins from Tables 1, 2, 5, and 6; wherein differential expression of the genes in Tables 1, 2, 5, and 6 is indicative of at least one toxic effect.

87. A method of predicting the progression of a toxic effect of a compound, comprising:
(a) detecting the level of expression in a tissue or cell sample exposed to the compound often or more genes or proteins from Tables 1, 2, 5, and 6; wherein differential expression of the genes in Tables 1, 2, 5, and 6 is indicative of toxicity progression.

88. A method of predicting the hepatotoxicity of a compound, comprising:
(a) detecting the level of expression in a tissue or cell sample exposed to the compound often or more genes or proteins from Tables 1, 2, 5, and 6; wherein differential expression of the genes in Tables 1, 2, 5, and 6 is indicative of hepatotoxicity.

89. A method of identifying an agent that modulates the onset or progression of a toxic response, comprising:
(a) exposing a cell to the agent and a known toxin; and
(b) detecting the expression level often or more genes or proteins from Tables 1, 2, 5, and 6; wherein differential expression of the genes in 1, 2, 5, and 6 is indicative of toxicity.

90. A method of predicting the cellular pathways that a compound modulates in a cell, comprising:
(a) detecting the level of expression in a tissue or cell sample exposed to the compound often or more genes or proteins from Tables 1, 2, 5, and 6; wherein differential expression of the genes in Tables 1, 2, 5, and 6 is associated the modulation of at least one cellular pathway.

91. A method of predicting at least one toxic effect of a test compound, comprising:
(a) preparing a gene expression profile from a liver cell or tissue sample exposed to the test compound; and
(b) comparing the gene expression profile to a database comprising quantitative gene expression information for at least ten genes, gene fragments, or proteins of Tables 1, 2, 5, and 6 from a liver cell or tissue sample that has been exposed to at least one toxin and quantitative gene expression information for at least ten genes, gene fragments, or proteins of Tables 1, 2, 5, and 6 from a control liver cell or tissue sample exposed to the toxin excipient, thereby predicting at least one toxic effect of the test compound.

92. The method of claim 86, wherein the proteins are secreted proteins.
